(19) United States
(12) Patent Application Publication
(10) Pub. No.: US 2023/0383326 A1
(43) Pub. Date: Nov. 30, 2023

(54) CELLULOSE SACCHARIFICATION METHOD

(71) Applicant: SEIKO EPSON CORPORATION, Tokyo (JP)

(72) Inventors: Sayaka YAMASAKI, Chino (JP); Hideki TANAKA, Chino (JP); Yoshiki NAKASHIMA, Suwa (JP); Yoko NAKAI, Chino (JP)

(21) Appl. No.: 18/324,416

(22) Filed: May 26, 2023

(30) Foreign Application Priority Data
May 30, 2022 (JP) 2022-087479

(51) Int. Cl. C12P 19/14; C12P 19/04

(52) U.S. Cl. CPC C12P 19/14; C12P 19/04

(57) ABSTRACT

A cellulose saccharification method includes a sterilization step of feeding, into a saccharification tank, an acidic liquid having a pH of 4.0 or less and a raw material including cellulose, and immersing, at a pH of 4.0 or less, the raw material in the acidic liquid for 30 minutes or longer; a mixing step of feeding water into the saccharification tank to obtain a liquid mixture having a pH of 4.4 or more; and a saccharification step of adding an enzyme to the liquid mixture and causing a saccharification reaction to proceed through stirring.

The present application is based on, and claims priority from JP Application Serial Number 2022-087479, filed May 30, 2022, the disclosure of which is hereby incorporated by reference herein in its entirety.

### BACKGROUND

### 1. Technical Field

The present disclosure relates to a cellulose saccharification method.

### 2. Related Art

Saccharification of biomass such as cellulose to obtain glucose has been attracting attention, for example, from the viewpoints of so-called carbon minus, efficient use of biomass, and application to bioethanol. For example, JP-T-2014-003912 discloses a method for producing a sugar through saccharification of biomass as a raw material. In the method disclosed in JP-T-2014-003912, biomass is sterilized by acid treatment, is subsequently made into a slurry, and is allowed to undergo a saccharification reaction.

In one cellulose saccharification method, cellulose is saccharified by introducing, into a saccharification tank, a raw material and a saccharification reaction liquid including an enzyme and causing an enzyme reaction to proceed through stirring. In this method, a sugar obtained through saccharification may be consumed by microorganisms and the like derived from the raw material; thus, the raw material is sterilized in some cases. However, in the sterilization method disclosed in JP-T-2014-003912, biomass is subjected to sterilization treatment in a container different from a saccharification device, and the biomass after sterilization treatment is introduced into the saccharification device by using a transfer unit and is subjected to a saccharification reaction. Such a saccharification device requires a separate device for sterilization treatment, leading to an increase in the size of the system as a whole.

Accordingly, there is a need for a cellulose saccharification method that is relatively small in scale and efficient and requires no dedicated equipment for sterilization of raw materials.

### SUMMARY

According to an aspect of the present disclosure, there is provided a cellulose saccharification method including a sterilization step of feeding, into a saccharification tank, an acidic liquid having a pH of 4.0 or less and a raw material including cellulose, and immersing, at a pH of 4.0 or less, the raw material in the acidic liquid for 30 minutes or longer; a mixing step of feeding water into the saccharification tank to obtain a liquid mixture having a pH of 4.4 or more; and a saccharification step of adding an enzyme to the liquid mixture and causing a saccharification reaction to proceed through stirring.

### BRIEF DESCRIPTION OF THE DRAWINGS

FIG. 1 is a schematic diagram illustrating an example of a saccharification tank according to an embodiment.

FIG. 2 is a flowchart illustrating an example of a saccharification method according to the embodiment.

### DESCRIPTION OF EXEMPLARY EMBODIMENTS

An embodiment of the present disclosure will be described hereinafter. The embodiment described below describes an example of the present disclosure. The present disclosure is not limited to the following embodiment, but encompasses various modifications embodied without changing the spirit of the present disclosure. Not all of the configurations described below are essential to the present disclosure.

A cellulose saccharification method according to the present embodiment includes a sterilization step of feeding, into a saccharification tank, an acidic liquid having a pH of 4.0 or less and a raw material including cellulose, and immersing, at a pH of 4.0 or less, the raw material in the acidic liquid for 30 minutes or longer; a mixing step of feeding water into the saccharification tank to obtain a liquid mixture having a pH of 4.4 or more; and a saccharification step of adding an enzyme to the liquid mixture and causing a saccharification reaction to proceed through stirring.

### 1. Sterilization Step

In the sterilization step, an acidic liquid having a pH of 4.0 or less and a raw material including cellulose are fed into a saccharification tank, and the raw material is immersed, at a pH of 4.0 or less, in the acidic liquid for 30 minutes or longer.

### 1.1. Saccharification Tank

The saccharification tank is not particularly limited as long as the raw material and the liquid can be introduced into the saccharification tank and can be stirred therein. The scale of the saccharification tank is also not limited and may be a laboratory scale such as a beaker or flask, a pilot plant scale, or a commercial plant scale.

The saccharification tank may include a container and a lid. The saccharification tank may include, as appropriate, an inlet for the raw material and the liquid, an outlet for the product, an interior stirring mechanism, a window for observation of the interior, a heating/cooling heater, a refrigerant pipe, a jacket, and other pipes. The saccharification tank may further include, for example, a liquid level gauge and/or a thermometer and may have an opening for installation thereof.

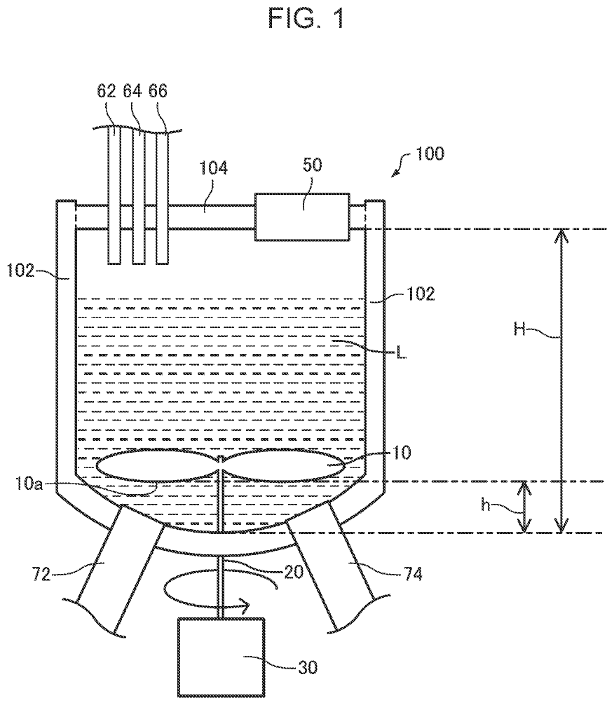

FIG. 1 is a diagram schematically illustrating a saccharification tank 100 which is an example of a saccharification tank that can be used to implement the saccharification method according to the present embodiment. In the example illustrated in FIG. 1, a mixture L containing a raw material has been fed into the saccharification tank 100. The mixture L is stirred with a stirring shaft 20 and a stirring blade 10 rotationally driven by a motor 30.

In the example illustrated in FIG. 1, the saccharification tank 100 includes a lid 104 closing an upper opening of a container 102. The lid 104 is provided with an inlet 50 through which the raw material is fed into the saccharification tank 100, a pipe 62 through which an enzyme is introduced, a pipe 64 through which a surfactant is introduced, and a pipe 66 through which a pH adjuster is introduced. A collection line 72 through which the liquid after the reaction is collected from the saccharification tank 100 and a residue drain 74 through which residue is discharged after the liquid is discharged are provided at the lower portion of the container 102.

Materials resistant to corrosion by the acidic liquid are preferably used for members such as the saccharification tank and the stirring blade. Each member may be appropriately coated.

### 1.2. Raw Material

The raw material introduced into the saccharification tank includes cellulose. The raw material may include components other than cellulose. Examples of the components other than cellulose include components derived from wood, such as lignin and hemicellulose; and components of processed wood, such as fillers, pigments, resin components, clay, binders, toner, water, and oil.

Cellulose may be derived from paper, pulp, or the like. When paper is used as the raw material, the raw material is readily available. Paper may include printed waste paper. Examples of printed waste paper include copying paper, newspaper, and magazines. Printed waste paper is preferably used, for example, because environmental resources and underground resources are conserved and waste can be reduced.

Paper, waste paper, or the like may be introduced into the saccharification tank in a pulverized state. Pulverization may be conducted by, for example, cutting with a shredder or the like, or a dry defibration machine or the like may be used for pulverization. Pulverization may also be conducted, for example, by wet defibration. When the raw material is in a pulverized or coarsely crushed state, components other than cellulose included in the raw material are more easily isolated from cellulose, and cellulose more readily comes into contact with the liquid introduced into the saccharification tank. Consequently, the efficiency of at least one of the sterilization step, the mixing step, and the saccharification step can be improved. In addition, pulverized waste paper or the like has an increased surface area and can thus improve the efficiency of each step.

Although the raw material is sterilized in the sterilization step, the raw material may be pre-sterilized before the sterilization step. Examples of methods for such sterilization include high-pressure heated steam and ultraviolet irradiation. As a result, levels of microorganisms, bacteria, and the like derived from the raw material can be further reduced.

### 1.3. Acidic Liquid

A liquid having a pH of 4.0 or less is used as the acidic liquid. For example, an acid aqueous solution adjusted to a pH of 4.0 or less may be used as the acidic liquid. A buffer solution adjusted to a pH of 4.0 or less may also be used as the acidic liquid.

Examples of acids that can be used to adjust the pH of the acidic liquid include inorganic acids and organic acids. Examples of inorganic acids include hydrochloric acid, nitric acid, sulfuric acid, hydrofluoric acid, phosphoric acid, and boric acid. Examples of organic acids include acetic acid, propionic acid, butanoic acid, pentanoic acid, hexanoic acid, heptanoic acid, octanoic acid, nonanoic acid, decanoic acid, oxalic acid, maleic acid, methylmalonic acid, benzoic acid, p-aminobenzoic acid, p-toluenesulfonic acid, benzenesulfonic acid, trifluoroacetic acid, malonic acid, sulfonic acid, phthalic acid, fumaric acid, citric acid, tartaric acid, citraconic acid, malic acid, glutaric acid, glutamic acid, and aspartic acid. One of these acids may be used alone, or two or more thereof may be used in combination.

Examples of alkalis that can be used to adjust the pH of the acidic liquid include nitrogen-containing organic or inorganic basic compounds, hydroxides of alkali metals and group 2 metals, various carbonates and hydrogencarbonates, quaternary ammonium hydroxides and salts thereof, ammonia, and amines. Specific examples of hydroxides of alkali metals include potassium hydroxide and sodium hydroxide. Examples of hydroxides of group 2 metals include calcium hydroxide, strontium hydroxide, and barium hydroxide. Specific examples of carbonates and hydrogencarbonates include ammonium hydrogencarbonate, ammonium carbonate, potassium hydrogencarbonate, potassium carbonate, sodium hydrogencarbonate, and sodium carbonate. Specific examples of quaternary ammonium hydroxides and salts thereof include tetramethylammonium hydroxide (TMAH), tetraethylammonium hydroxide, and tetrabutylammonium hydroxide. Specific examples of amines include methylamine, dimethylamine, trimethylamine, ethylamine, diethylamine, triethylamine, ethylenediamine, monoethanolamine, N-(β-aminoethyl)ethanolamine, hexamethylenediamine, diethylenetriamine, triethylenetetramine, piperazine anhydride, piperazine hexahydrate, 1-(2-aminoethyl) piperazine, N-methylpiperazine, guanidine, and azoles such as imidazole and triazole. One of these alkalis may be used alone, or two or more thereof may be used in combination.

The acidic liquid may constitute a buffer solution. The acidic liquid has a pH of 4.0 or less and may have a buffering effect, and known buffer solutions can be used. Even when a buffer solution is used, the above-described acids, alkalis, and the like may be used. For example, a buffer solution including hydrochloric acid/potassium chloride, tartaric acid, citric acid, glycine, formic acid, acetic acid, succinic acid, phosphoric acid, or a salt thereof can be used as the buffer solution, and a buffer solution including triethanolamine, tris(hydroxymethyl)aminomethane, diethanolamine, boric acid, ammonia, carbonic acid, or a salt thereof can be used as a basic buffer solution.

The pH of the acidic liquid can be measured, for example, by using a pH meter in a manner in which a glass electrode after three-point calibration with a standard buffer solution is placed in the liquid to be measured, and a stabilized value after a lapse of at least two minutes is measured. For example, a LAQUA (registered trademark) manufactured by HORIBA, Ltd., a microsampling pH monitor (e.g., UP-100), or a similar product can be used as the pH meter. A phthalate pH buffer solution having a pH of 4.01 (at 25° C.), a neutral phosphate pH buffer solution having a pH of 6.86 (at 25° C.), a carbonate pH buffer solution having a pH of 10.01 (at 25° C.), or the like can be used as the standard buffer solution.

The pH of the acidic liquid is not particularly limited as long as the pH is 4.0 or less. Levels of microorganisms, bacteria, and the like in the raw material can be reduced by the action of the acidic liquid. The pH of the acidic liquid is preferably 1.5 or more and less than 4.0, more preferably 2.0 or more and 3.8 or less, and further preferably 2.5 or more and 3.5 or less. When the pH of the acidic liquid is too low, the saccharification tank may be corroded. The pH of the acidic liquid can be set within a range in which the saccharification tank is not corroded.

The acidic liquid contains water as a main component. Industrial water, groundwater, filtered water, tap water, or the like may be used as water without particular limitations. However, pure water such as ion-exchange water, ultrafiltration water, reverse osmosis water, or distilled water, or water from which ionic impurities have been removed as much as possible, such as ultrapure water, is preferably used. In addition, water sterilized by ultraviolet irradiation, addition of hydrogen peroxide, or the like is preferably used because growth of fungi and bacteria during and after an enzyme reaction can be suppressed when the liquid is stored for a long period.

### 1.4. Immersion

In the sterilization step, the raw material is immersed at a pH of 4.0 or less in the acidic liquid for 30 minutes or longer. In the sterilization step, the raw material is immersed in the acidic liquid within the saccharification tank, thereby sterilizing the raw material. The raw material may be fed after introducing the acidic liquid into the saccharification tank and vice versa. The raw material may be fed into the saccharification tank while spraying the acidic liquid into the raw material to immerse the raw material in the acidic acid.

Stirring may be carried out inside the saccharification tank during at least one of the time periods before, during, and after feeding of the raw material and the acidic liquid into the saccharification tank, if needed.

In the sterilization step, the state where the pH of the acidic liquid is 4.0 or less is maintained for 30 minutes or longer. When the pH of the acidic liquid exceeds 4.0 during the sterilization step, the pH is controlled to 4.0 or less by, for example, additionally feeding the acidic liquid or adding an acid. In the sterilization step, the immersion is carried out such that the total time period during which the pH of the acidic liquid is 4.0 or less is 30 minutes or longer.

For example, when waste paper is used as the cellulose raw material, the pH may increase as a neutralization reaction occurs due to the presence of a filler or the like during the sterilization step; however, the pH can be decreased to a value suitable for sterilization, that is, to 4.0 or less, by further feeding the acidic liquid and/or an acid.

The pH of the acidic liquid during immersion is preferably 1.5 or more and less than 4.0, more preferably 2.0 or more and 3.8 or less, and further preferably 2.5 or more and 3.5 or less.

The immersion time is not particularly limited as long as it is 30 minutes or longer, but is preferably 40 minutes or longer, more preferably 50 minutes or longer, and further preferably one hour or longer. The upper limit of the immersion time is, for example, within one day but is not particularly limited thereto and is appropriately set in consideration of the entire cellulose saccharification process and the throughput.

The amounts of the acidic liquid and the raw material fed into the saccharification tank during the sterilization step are appropriately set according to the scale and performance of the saccharification tank. Although the mixing ratio of the raw material to the acidic liquid can also be appropriately set, the amount of cellulose is, for example, 50 parts by mass and more preferably 30 parts by mass based on 100 parts by mass of the acidic liquid.

The pH of the acidic liquid is measured in the sterilization step. When bubbles are generated in the acidic liquid, it is more preferable that the pH of the acidic liquid be measured after generation of bubbles ends. In this manner, the pH of the acidic liquid can be more accurately measured, and the sterilization step can be conducted more effectively.

### 2. Mixing Step

In the mixing step, water is fed into the saccharification tank to prepare a liquid mixture having a pH of 4.4 or more. In the mixing step, water is added to the mixture after the sterilization step to prepare a liquid mixture having a pH of 4.4 or more. The water to be added to the saccharification tank in the mixing step is similar to the water used in the sterilization step described above.

In the mixing step, the pH may be set to 4.4 or more through addition of water, but a basic liquid may be additionally fed. This allows the pH to be easily set to 4.4 or more. The basic liquid used in this case is not limited as long as it is an alkaline liquid. For example, alkaline aqueous solutions, buffer solutions, and the like as described above can be used.

In the mixing step, a weakly acidic pH condition optimal for the enzyme reaction can be achieved by adding water. In addition, when the pH does not increase sufficiently, a basic liquid (such as NaOH aq.) can be added, as needed, to achieve a pH suitable for the saccharification reaction in the saccharification step.

The pH of the liquid mixture is measured in the mixing step. When bubbles are generated in the liquid mixture, it is more preferable that the saccharification step described later be started after generation of bubbles ends. In this manner, the contact between cellulose and an enzyme can be improved, thereby improving the saccharification reaction efficiency.

### 2.2 pH of Liquid Mixture

The pH of the liquid mixture obtained in the mixing step may be 4.4 or more but may be adjusted according to the optimal pH for the enzyme used in the saccharification step. For example, when the enzyme used is Cellic CTec2 (manufactured by Novozymes A/S), the pH of the saccharification reaction liquid is 4.5 or more and 6.0 or less and is preferably 5.0 or more and 5.7 or less. The pH may be adjusted by adding, for example, an acid, an alkali, or a pH adjuster.

The pH can be adjusted by adding, to the liquid mixture, sodium acetate, acetic acid, sulfuric acid, a sodium hydroxide aqueous solution, or the like. In a configuration in which the pH of the liquid mixture can be monitored, the pH may be adjusted during the mixing step and/or the saccharification step. For example, when the pH exceeds a predetermined pH during stirring inside the saccharification tank, acetic acid, sulfuric acid, or the like is added, whereas when the pH drops below a predetermined pH, a sodium hydroxide aqueous solution or the like is added.

### 3. Saccharification Step

In the saccharification step, an enzyme is added to the liquid mixture, and a saccharification reaction is caused to proceed through stirring.

### 3.1. Enzyme

An enzyme that functions to decompose cellulose into a sugar by cleaving β-1,4-glucoside bonds can be used as the enzyme. Examples of cellulolytic enzymes include endoglucanase, cellobiohydrolase, and cellobiase (R-glucosidase). More specific examples of cellulolytic enzymes include Cellulase SS (manufactured by Nagase ChemteX Corporation), Accellerase Duet (manufactured by Genencor), Cellic CTec2 (manufactured by Novozymes A/S), Cellic CTec3 (manufactured by Novozymes A/S), and Meilase (manufactured by Meiji Seika Pharma Co., Ltd.), and two or more of these enzymes may be used in combination. In addition, xylanase may be included to simultaneously decompose xylan present on the surface of cellulose and enhance saccharification efficiency.

The enzyme may be introduced into the saccharification tank as a powder, a solution, or a dispersion. In addition, the enzyme may be additionally introduced into the saccharification tank as needed.

### 3.2. Temperature

The temperature of the liquid mixture in the saccharification step is preferably adjusted to the optimal temperature for the enzyme used. For example, when the enzyme used is Cellic CTec2 (manufactured by Novozymes A/S), the temperature of the saccharification reaction liquid is ° C. or more and 57° C. or less, preferably 45° C. or more and ° C. or less, and more preferably 49° C. or more and 50° C. or less.

The temperature of the liquid in the saccharification tank is adjusted using a heater, a cooler, a controller, or the like, as appropriate.

### 3.3. Stirring

In the saccharification step, a cellulose-derived sugar is produced through stirring inside the saccharification tank. The saccharification step is conducted for two hours or more and within one week, although it depends on the enzyme performance and the overall scale. The time period for the saccharification step is typically ten hours or more and within five days, more preferably one day or more and within four days, and further preferably two days or more and within three days.

Stirring in the saccharification step is carried out by the appropriate stirring mechanism as described above. The rotational speed of the motor and other conditions during stirring can be set, as appropriate, according to, for example, the scale and configuration of the saccharification tank and the shape of a stirring bar or stirring blade. If possible, stirring may be appropriately carried out during the sterilization step and the mixing step on an as-needed basis.

Stirring is carried out through rotation of a stirring blade. Stirring is carried out using a stirring mechanism having a stirring blade within the saccharification tank.

Examples of the stirring mechanism include a magnetic stirrer and a stirring bar; a motor for stirring, a shaft, and a stirring blade; and combinations thereof. The stirring mechanism can be appropriately selected according to the scale and the material stirring efficiency.

The stirring blade is preferably disposed at a bottom portion of the saccharification tank. That is, it is more preferable that the lowest portion of the stirring blade be close to the lowest portion of the internal space within the saccharification tank. For example, when the distance between the lowest portion of the internal space within the saccharification tank and the highest portion of the internal space within the saccharification tank is taken as 1, the distance between the lowest portion of the stirring blade and the lowest portion of the internal space within the saccharification tank is preferably 0.3 or less, more preferably 0.2 or less, and further preferably 0.1 or less.

In the saccharification tank 100 illustrated in FIG. 1, the ratio (h/H) is about 0.17, where H is the distance between the lowest portion of the internal space within the saccharification tank 100 and the highest portion of the internal space within the saccharification tank 100, and h is the distance between the lowest portion 10a of the stirring blade 10 and the lowest portion of the internal space within the saccharification tank 100.

### 4. Modifications and the Like

In the saccharification method according to the present embodiment, a surfactant may be used in at least one of the sterilization step, the mixing step, and the saccharification step to enhance the wettability and affinity of the liquid for the raw material, if needed. Any surfactant that does not inhibit the enzyme reaction can be used without particular limitations. The introduction of a surfactant into the saccharification tank allows the acidic liquid to more readily wet the raw material, thereby improving the saccharification reaction efficiency.

When a surfactant is used, it is more preferable that the surfactant include a surfactant having a defoaming effect. Surfactants having a defoaming effect may be referred to as defoaming agents. Examples of defoaming agents include silicone-based defoaming agents, polysiloxane-based defoaming agents, acetylene glycol-based defoaming agents, polyether-based defoaming agents, and aliphatic acid ester-based defoaming agents, but are not particularly limited thereto.

Examples of commercially available silicone-based surfactants include three-dimensional siloxane “FOAM BAM (registered trademark) MS-575” (product name, manufactured by MUNZING Chemie GmbH), KM-71 and KM-75 (product names, manufactured by Shin-Etsu Chemical Co., Ltd.), and BYK-093 and BYK-094 (product names, manufactured by BYK).

Examples of commercially available polysiloxane-based defoaming agents include KM-73A, KM-73E, KM-71, KM-85, KM-89, KM-98, KM-7752, KS-531, KS-540, KS-530, KS-537, and KS-538 (product names, manufactured by Shin-Etsu Chemical Co., Ltd.), BYK-020, BYK-021, BYK-022, BYK-023, BYK-024, BYK-044, and BYK-094 (product names, manufactured by BYK), and TSA6406, TSA780, TSA739, and TSA775 (product names, manufactured by Momentive Performance Materials Japan LLC).

Examples of acetylene glycol-based defoaming agents include 2,4,7,9-tetramethyl-5-decyne-4,7-diol, alkylene oxide adducts of 2,4,7,9-tetramethyl-5-decyne-4,7-diol, 2,4-dimethyl-5-decyn-4-ol, and alkylene oxide adducts of 2,4-dimethyl-5-decyn-4-ol. Examples of commercially available acetylene glycol-based defoaming agents include OLFINE 104 series and E series such as OLFINE E1010 (product names, manufactured by Air Products and Chemicals, Inc.) and SURFYNOL 465, 61, and DF110D (product names, manufactured by Nissin Chemical Industry Co., Ltd.).

Inclusion of the defoaming agent in the saccharification reaction liquid results in less foaming and facilitates defoaming upon foaming, and can thus further suppress, for example, spilling from the saccharification tank and clogging of pipes.

The saccharification reaction liquid may include a surfactant other than the surfactants described as defoaming agents above. Examples of such a surfactant include, but are not limited to, silicone-based surfactants, polyoxyethylene alkyl ether-based surfactants, polyoxypropylene alkyl ether-based surfactants, polycyclic phenyl ether-based surfactants, sorbitan derivatives, fluorinated surfactants, and nonionic surfactants.

### 5. Other Steps

The saccharification method according to the present embodiment may include, for example, a pulverization step, a collection step, and a cleaning step in addition to the above-described steps.

### 5.1. Pulverization Step

The pulverization step is a step of pulverizing the raw material before introduction into the saccharification tank. The method of pulverizing the raw material is not particularly limited, and a method such as cutting with a shredder or the like or pulverization (defibration) with a dry defibration machine or the like can be employed. The raw material is preferably pulverized by a dry process, but may be pulverized, for example, by wet defibration.

When the raw material is in a pulverized state, components other than cellulose included in the raw material are more easily isolated from cellulose, and cellulose more readily comes into contact with the liquid. Consequently, the efficiency of at least one of the sterilization step, the mixing step, and the saccharification step can be improved.

### 5.2 Collection Step

The saccharification method may include a step of collecting the liquid from the saccharification tank after the saccharification step. The collection step may be carried out through a pipe or the like. The collected liquid including a sugar may be filtered.

### 5.3 Cleaning of Saccharification Tank

The saccharification method may include a step of cleaning the saccharification tank after the saccharification step. The step of cleaning the saccharification tank may be, for example, a step of removing precipitate remaining in the saccharification tank, a step of removing oil remaining in the saccharification tank, and/or a step of cleaning the stirring mechanism, before and/or after the collection step.

### 6. Advantageous Effects

In the saccharification method according to the present embodiment, sterilization treatment can be conducted within the saccharification tank by immersing the raw material including cellulose in the acidic liquid within the saccharification tank, and a pretreatment process before the saccharification reaction can thus be simplified. Furthermore, since the pH can be brought close to the optimal pH for the enzyme reaction by directly diluting the liquid mixture with water, adjustment of the pH of the liquid mixture can be simplified.

### 7. Example of Flow of Saccharification Method

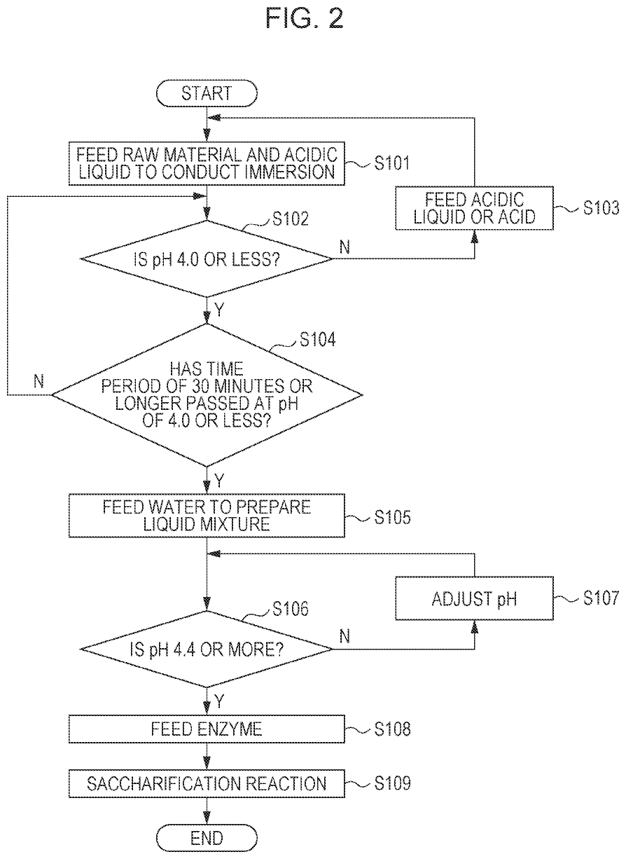

FIG. 2 is a flowchart illustrating an example of the saccharification method according to the present embodiment. A description is given with reference to FIG. 2.

First, the raw material and the acidic liquid are fed into the saccharification tank to conduct immersion (S101). Then, the pH of the acidic liquid is measured, and it is determined whether the pH is 4.0 or less (S102). When the pH exceeds 4.0 (N in S102), the acidic liquid and/or an acid is fed into the saccharification tank (S103). The pH of the acidic liquid is measured, and it is determined whether the pH is 4.0 or less. When the pH is 4.0 or less (Y in S102), it is determined whether a time period of 30 minutes or longer has passed at a pH of 4.0 or less (S104). When the pH has been 4.0 or less for less than 30 minutes (N in S104), it is determined whether the pH is 4.0 or less (S102), and the system is left to wait. When the pH has been 4.0 or less for 30 minutes or longer (Y in S104), water is fed to prepare a liquid mixture (S105).

Here, the process from the step (S101) of feeding the raw material and the acidic liquid to conduct immersion to the time at which it is determined that the pH has been 4.0 or less for 30 minutes or longer (Y in S104) corresponds to the above-described sterilization step.

After water is fed to prepare a liquid mixture (S105), the pH of the mixture is measured, and it is determined whether the pH is 4.4 or more (S106). When the pH is less than 4.4 (N in S106), the pH is adjusted until the pH becomes 4.4 or more. When the pH of the mixture is 4.4 or more (Y in S106), the enzyme is fed (S108) to start a saccharification reaction (S109).

Here, the process from the step (S105) of feeding water to prepare a liquid mixture to the time at which the pH is measured to be 4.4 or more (Y in S106) corresponds to the above-described mixing step. Then, the saccharification reaction step (S109) corresponds to the above-described saccharification step. Although not illustrated in the flowchart, stirring is carried out inside the saccharification tank at least in the saccharification step.

Since the saccharification method according to the present embodiment includes the above-described flow, no device configuration for sterilization is required, a small-scale system can be constructed, and the pH can be easily adjusted.

The above-described embodiment and modifications are merely examples, and the present disclosure is not limited thereto. For example, the embodiment and modifications can each be combined, as appropriate, with one another.

The present disclosure encompasses configurations substantially identical to the configuration described in the embodiment, for example, configurations with the same function, method, and result or configurations with the same purpose and effect. The present disclosure also encompasses configurations in which a portion not essential for the configuration described in the embodiment is replaced. The present disclosure also encompasses configurations which provide the same advantageous effects as the configuration described in the embodiment or with which the same purpose as the configuration described in the embodiment can be achieved. The present disclosure also encompasses configurations in which a known feature is added to the configuration described in the embodiment.

The following aspects are derived from the above-described embodiment and modifications.

A cellulose saccharification method includes a sterilization step of feeding, into a saccharification tank, an acidic liquid having a pH of 4.0 or less and a raw material including cellulose, and immersing, at a pH of 4.0 or less, the raw material in the acidic liquid for 30 minutes or longer; a mixing step of feeding water into the saccharification tank to obtain a liquid mixture having a pH of 4.4 or more; and a saccharification step of adding an enzyme to the liquid mixture and causing a saccharification reaction to proceed through stirring.

According to this cellulose saccharification method, sterilization treatment can be conducted within the saccharification tank by immersing cellulose in the acidic liquid within the saccharification tank, and the pretreatment process can thus be simplified. Furthermore, since the pH can be brought close to the optimal pH for the enzyme reaction by directly diluting the liquid mixture with water, adjustment of the pH of the liquid mixture can be simplified.

In the cellulose saccharification method described above, the pH may be measured in the sterilization step, and when the pH exceeds 4.0, the acidic liquid may be further fed such that the pH becomes 4.0 or less.

According to this cellulose saccharification method, although the pH may increase as a neutralization reaction occurs due to the presence of a filler or the like during the sterilization step when waste paper is used as the cellulose raw material, the pH can be decreased to a value suitable for sterilization by further feeding the acidic liquid.

In the cellulose saccharification method described above, a basic liquid may be fed in the mixing step.

According to this cellulose saccharification method, a weakly acidic pH optimal for the enzyme reaction can be achieved by adding water during the mixing step, whereas, when the pH does not increase sufficiently, a basic liquid (such as NaOH aq.) can be added, as needed, to achieve a pH suitable for the saccharification reaction.

In the cellulose saccharification method described above, when bubbles are generated in the liquid mixture in the mixing step, the saccharification step may be started after generation of bubbles ends.

According to this cellulose saccharification method, the contact between cellulose and the enzyme can be improved, thereby improving the saccharification reaction efficiency.

In the cellulose saccharification method described above, the raw material may be waste paper.

According to this cellulose saccharification method, the raw material is readily available.

In the cellulose saccharification method described above, the stirring may be carried out with a stirring blade disposed at a bottom portion of the saccharification tank.

According to this saccharification method, the stirring efficiency during the stirring step and the sterilization step can be enhanced by disposing the stirring blade at the bottom portion of the saccharification tank, allowing a liquid to more easily permeate the cellulose raw material and thus further enhancing the efficiency of both of the saccharification reaction and mixing.

What is claimed is:

1. A cellulose saccharification method comprising:
a sterilization step of feeding, into a saccharification tank, an acidic liquid having a pH of 4.0 or less and a raw material including cellulose, and immersing, at a pH of 4.0 or less, the raw material in the acidic liquid for 30 minutes or longer;
a mixing step of feeding water into the saccharification tank to obtain a liquid mixture having a pH of 4.4 or more; and
a saccharification step of adding an enzyme to the liquid mixture and causing a saccharification reaction to proceed through stirring.

2. The cellulose saccharification method according to claim 1, wherein
pH is measured in the sterilization step, and when the pH exceeds 4.0, the acidic liquid is further fed such that the pH becomes 4.0 or less.

3. The cellulose saccharification method according to claim 1, wherein
a basic liquid is fed in the mixing step.

4. The cellulose saccharification method according to claim 1, wherein
when bubbles are generated in the liquid mixture in the mixing step, the saccharification step is started after generation of bubbles ends.

5. The cellulose saccharification method according to claim 1, wherein
the raw material is waste paper.

6. The cellulose saccharification method according to claim 1, wherein
the stirring is carried out with a stirring blade disposed at a bottom portion of the saccharification tank.
